(19) United States
(12) Patent Application Publication
(10) Pub. No.: US 2015/0202203 A1
(43) Pub. Date: Jul. 23, 2015

(54) Method of Treating Gastrointestinal Stromal Tumors

(71) Applicants: John E Monahan, Walpole, MA (US); Fang Li, Arlington, MA (US)

(72) Inventors: John E Monahan, Walpole, MA (US); Fang Li, Arlington, MA (US)

(73) Assignee: Novartis AG, Basel (CH)

(21) Appl. No.: 14/413,045

(22) PCT Filed: Apr. 12, 2013
(86) PCT No.: PCT/US13/36273

§ 371 Date: Jan. 6, 2015

Related U.S. Application Data
(60) Provisional application No. 61/670,168, filed on Jul. 11, 2012.

(30) Foreign Application Priority Data
Jul. 11, 2012 (US) PCT/US2012/061535

(51) Int. Cl. A61K 31/506; A61K 31/496

(52) U.S. Cl. CPC A61K 31/506; A61K 31/496

(57) ABSTRACT

The present invention relates to a method of treating gastrointestinal stromal tumors (GIST), especially GIST, which is progressing after imatinib therapy or after imatinib and sunitinib therapy, using a combination comprising (a) a c-kit inhibitor and (b) a dual KIT inhibitor and FGFR inhibitor or FGFR inhibitor.

The present invention relates to a method of treating gastrointestinal stromal tumors (GIST) in a human patient population using a combination comprising (a) a c-kit inhibitor and (b) a PI3K inhibitor or FGFR inhibitor.

GIST are the most frequent mesenchymal tumors of the gastrointestinal tract. These tumors are thought to arise from the interstitial cells of Cajal, which compose the myenteric plexus found in the stomach and bowel. Primary GIST most frequently occur in the stomach (50-60%), small bowel (20-30%), and large bowel (10%), with the esophagus, mesentery, omentum, and retroperitoneum accounting for the remaining cases. On the basis of population-based incidence rates in Sweden, it has been estimated that approximately 5000 new cases of GIST are diagnosed each year in the US. GIST predominantly occur in middle-aged and older people, with a median onset age of approximately 60 years and no apparent gender preference.

GIST may display a variety of phenotypic features, many of which correlate with patient prognosis. Thus, a consensus meeting emphasized tumor size and mitotic index for risk stratification of primary GIST, with such risk being correlated with tumor recurrence. At the present time, risk stratification based on pathologic criteria is preferable to the use of such terms as benign or malignant GIST. Patients with primary gastric GIST seem to fare slightly better than those with intestinal tumors. GIST have a tendency to recur both locally and in the form of peritoneal and liver metastases, with lymph-node metastases being infrequent. Surgical resection is the mainstay of therapy for primary GIST, and the disease is typically refractory to cytotoxic chemotherapy. The diagnosis of GIST has been facilitated by the dis-covery that these tumors stain positively with an immunohistochemical marker (CD117) pre-viously used to stain the interstitial cells of Cajal. The antibody used in the immunohistochemical reaction recognizes the extracellular domain of the stem-cell factor receptor, KIT. Currently, KIT expression is a major diagnostic criterion for GIST, and few other KIT-positive mesenchymal tumors of the gastrointestinal tract are likely to be confused with GIST; notable exceptions include metastatic melanoma and malignant vascular tumors. Approximately 95% of GIST stain positively for CD117. In most of these cases, somatic mutations can be found in the gene encoding the KIT protein, typically in exons 11, 9 and 13. These mutations confer a gain of function to the receptor, which becomes constitutively activated regardless of the presence of ligand.

The mainstay of therapy for patients with primary GIST is surgical resection. However, surgery alone is generally not curative; the 5-year disease specific survival is reported to be 54%. Recurrence rates exceeding 50% within 2 years of resection of primary GIST and ap-proximating 90% after re-excision, underscored the need for effective postoperative treatment.

Imatinib received worldwide approval for the treatment of adult patients with KIT-positive (CD117) and unresectable and/or metastatic GIST and dramatically changed the prognosis for such patients by prolonging the overall and the progression-free-survival (PFS) and increasing the 5-year survival rate. Imatinib at doses ranging from 400 mg/day to 800 mg/day is used worldwide for the treatment of patients with unresectable and/or metastatic KIT-positive GIST. In addition, imatinib 800 mg/day significantly improves progression-free survival (PFS) in patients with advanced GIST harboring KIT exon 9 mutations compared to 400 mg/day.

As a result of the efficacy of imatinib for the treatment of patients with unresectable and/or metatastatic GIST, a double-blind, randomized phase III study (ACOSOGZ9001) was con-ducted to determine whether adjuvant treatment of adult patients with GIST following complete resection with 400 mg/day of imatinib for 12 months improved recurrence-free survival (RFS) compared with placebo. The results of the study indicated that treatment with imatinib significantly prolonged RFS. Based upon these data, imatinib at a dose of 400 mg/day was approved worldwide for adjuvant treatment of adult patients following resection of GIST. Results from SSGXVIII/AIO, a Phase III multicenter, open-label, randomized study to assess the efficacy and safety of 400 mg imatinib once daily over 12 months or 36 months in GIST patients following surgery, and who were estimated to be at high risk of disease recurrence are now available. The study data confirm that 36 months of adjuvant therapy with imatinib is well tolerated and superior to 12 months in prolonging RFS and overall survival in patients with GIST following surgical resection.

Despite the efficacy of imatinib, the treatment of metastatic GIST remains an area of unmet medical need with more than 50% of patients with advanced GIST progressing after 2 years of imatinib first line therapy.

Nilotinib is a KIT inhibitor which inhibits growth of GIST-T1 cells. However, the efficacy of nilotinib is significantly decreased in the presence of FGF2. Dovtinib (4-amino-5-fluoro-3-[5-(4-methylpiperazin-1-yl)-1H-benzimidazol-2-yl]quinolin-2(1H)-one) is a dual KIT inhibitor and FGFR inhibitor that that inhibits growth of GIST-T1 cells to the same level of maximum inhibition in the presence or absence of added FGF2 (FIG. 5).

Based on the finding that FGFR pathway could be a survival pathway in GISTs, combining a KIT inhibitor and a dual KIT inhibitor and FGFR inhibitor that target the survival pathways in GIST can produce a greater therapeutic effect than that obtained by administration of a KIT inhibitor alone.

As shown herein, the FGF2 growth factor and its receptor FGFR1 are over-expressed in primary GIST tissue, suggesting that FGFR pathway could be a survival pathway activated in GIST. FGFR1, but not FGF2, is over-expressed in GIST cell lines. However, the FGFR signaling pathway is activated in GIST cell lines in the presence of exogenous FGF2. In addition, GIST cell lines are less sensitive to the treatment of KIT inhibitors in the presence of added FGF2. Combination of FGFR inhibitors with KIT inhibitors produces strong synergistic activity and significantly improved efficacy in the presence of FGF2 in GIST cells, suggesting that a combination comprising an FGFR inhibitor and a KIT inhibitor can improve the efficacy of the current treatment strategies in GIST.

In a broader sense, the present invention provides a method of treating GIST, preferably GIST not harboring any KIT mutations, including KIT mutations and KIT resistant muttions by administering to a patient in need thereof a therapeutically effective amount of a FGFR inhibitor.

Furthermore, based on observations in GIST cell lines it was now surprisingly found that patients with GIST progressing after imatinib first line therapy, might be treated successfully with a combination comprising (a) a c-kit inhibitor and (b) a dual KIT inhibitor and FGFR inhibitor.

Furthermore, it is concluded that patients with GIST progressing after consecutive therapy with imatinib and sunitinib can be treated successfully with a combination comprising (a) a c-kit inhibitor and (b) a dual KIT inhibitor and FGFR inhibitor.

Hence, the present invention provides a method for treating GIST in a human patient progressing after imatinib therapy or consecutive imatinib and sunitinib therapy, comprising co-administration to said patient, e.g., concomitantly or in sequence, of a therapeutically effective amount of (a) a c-kit inhibitor and (b) a dual KIT inhibitor and FGFR inhibitor or FGFR inhibitor. More broadly, the present invention provides a method for treating GIST in a human patient in need thereof, comprising co-administration to said patient, e.g., concomitantly or in sequence, of a therapeutically effective amount of (a) a c-kit inhibitor and (b) a dual KIT inhibitor and FGFR inhibitor or FGFR inhibitor.

In a further aspect, the present invention relates to the use of a combination comprising (a) a c-kit inhibitor and (b) a dual KIT inhibitor and FGFR inhibitor or FGFR inhibitor for the manufacture of a medicament for the treatment of GIST, especially GIST progressing after imatinib first line therapy.

A further aspect of the invention relates to a combination comprising (a) a c-kit inhibitor and (b) a dual KIT inhibitor and FGFR inhibitor or FGFR inhibitor for the treatment of GIST, especially GIST progressing after imatinib therapy or GIST progressing after imatinib and sunitinib therapy.

### SHORT DESCRIPTION OF THE FIGURES

FIG. 1: FGF2 and FGFR1 are highly expressed in primary GISTs. Raw data (CEL files) of the expression profiles for 30,094 primary tumors were normalized by MAS5 algorithm using 150 as the target value.

FIG. 2: FGF2 expression is substantially higher in KIT-positive primary gastrointestinal stromal tumors (GISTs) than in other human primary tumor tissues. GAPDH Western blot is shown as a loading control.

FIG. 3: FGFR pathway is activated in GIST cell lines in the presence of various concentrations of added FGF2. FRS2 Tyr-Phosphorylation was used as the readout of FGFR signaling activation and measured by Western blot in the GIST cell lines. Total FRS2 level is shown as the loading control.

FIG. 4: KIT inhibition of Imatinib and dovitinib measured by Western blot in the GIST cell lines.

FIG. 5: GIST-T1 cells are less sensitive to nilotinib in the presence of added FGF2 than in the absence of added FGF2, and dovitinib restores maximum growth inhibition of GIST-T1 in the presence of FGF2, as compared to nilotinib.

FIG. 6: GIST882 cells are less sensitive to nilotinib in the presence of added FGF2 than in the absence of added FGF2, and dovitinib restores maximum growth inhibition of GIST882 in the presence of FGF2, as compared to nilotinib.

FIG. 7: Combination effect of imatinib plus dovitinib in GIST-T1 (A) and GIST882 (B) in the absence and presence of 20 ng/ml FGF2. The left panels show the percent inhibition relative to DMSO-treated cells for each single agent and combination treatments. Increasing concentrations of imatinib (CGP057148B) are shown along the left column from bottom to top and increasing concentrations of dovitinib along the bottom row from left to right. The middle panels show the excess inhibition for each point in the left panels. Excess inhibition was determined based on the Loewe synergy model that measures the effect on growth relative to what should be expected if the two drugs only function additively. Positive numbers indicate synergy, and negative numbers antagonism. The right panels are the isobolograms that display the interactions between the two compounds. The straight lines connecting the doses of imatinib and dovitinib represent the additive effect. Curves that lie below and to the left of the straight lines represent synergism.

The expression “c-kit inhibitor” as used herein includes, but is not limited to, 4-(4-methylpiperazin-1-ylmethyl)-N-[4-methyl-3-(4-(pyridin-3-yl)pyrimidin-2-ylamino)phenyl]-benzamide (Imatinib), 4-methyl-3-[[4-(3-pyridinyl)-2-pyrimidinyl]amino]-N-[5-(4-methyl-1H-imidazol-1-yl)-3-(trifluoromethyl)phenyl]benzamide (Nilotinib), masitinib, sunitinib, sorafenib, regorafeinib, motesanib, and, respectively, the pharmaceutically acceptable salts thereof.

In a preferred embodiment the c-kit inhibitor employed is Imatinib. Imatinib is specifically disclosed in the patent applications U.S. Pat. No. 5,521,184, the subject-matter of which is hereby incorporated into the present application by reference. Imatinib can also be prepared in accordance with the processes disclosed in WO03/066613. For the purpose of the present invention, Imatinib is preferably applied in the form of its mono-mesylate salt. Imatinib mono-mesylate can be prepared in accordance with the processes disclosed in U.S. Pat. No. 6,894,051. Comprised by the present invention are likewise the corresponding polymorphs, e.g. crystal modifications, which are disclosed in U.S. Pat. No. 6,894,051.

In a further preferred embodiment of the method described herein the mono-mesylate salt of Imatinib is administered orally in dosage forms as described in U.S. Pat. No. 5,521,184, U.S. Pat. No. 6,894,051 or US 2005-0267125. The mesylate salt of Imatinib is marketed under the brand Glivec® (Gleevec®). A preferred oral daily dosage of Imatinib is 200-600 mg, in particular 400 mg/day, administered as a single dose or divided into multiple doses, such as twice daily dosing.

In one embodiment of the present invention, the c-kit inhibitor employed is Nilotinib. Nilotinib and the process for its manufacture are disclosed in WO 04/005281, which is hereby incorporated into the present application by reference. Pharmaceutically acceptable salts of Nilotinib are especially those disclosed in WO2007/015871. For the purpose of the present invention, Nilotinib is preferably applied in the form of its mono-hydrochloride monohydrate salt. WO2007/015870 discloses certain polymorphs of nilotinib and its pharmaceutically acceptable salts useful for the present invention.

In the embodiment of the method described herein the mono-hydrochloride salt of Nilotinib is administered orally in dosage forms as described in WO2008/037716. The mono-hydrochloride salt of Nilotinib is marketed under the brand Tasigna®. A preferred oral daily dosage of Nilotinib is 200-1200 mg, e.g. 800 mg, administered as a single dose or divided into multiple doses, such as twice daily dosing.

The expression “FGFR inhibitor” as used herein includes, but is not limited to

(a) brivanib, intedanib, E-7080, ponatinib, SU-6668 and AZD-4547.
(b) the compounds disclosed in WO2009/141386, and
(c) WO2006/000420 (including 3-(2,6-dichloro-3,5-dimethoxy-phenyl)-1-{6-[4-(4-ethyl-piperazin-1-yl)-phenylamino]-pyrimid-4-yl}-methyl-urea monophosphate, BGJ398). BGJ398 is a pan-FGFR kinase inhibitor inhibiting FGFR 1-3 (IC50 between 3 and 7 nM).

The dual KIT inhibitor and FGFR inhibitor of the pharmaceutical combinations of the present invention includes at least one RTK inhibitor compound selected from the group con-sisting of compounds of Formula I or a tautomer thereof, compounds of Formula II or a tautomer thereof, compounds of Formula III or a tautomer thereof, a pharmaceutically acceptable salt of the compound, a pharmaceutically acceptable salt of the tautomer, or a mixture thereof.

The dual KIT inhibitor and FGFR inhibitor compound may be selected from a compound of formula I, a tautomer of the compound, a salt of the compound, a salt of the tautomer, or a mixture thereof, wherein the compound of formula I has the following formula:

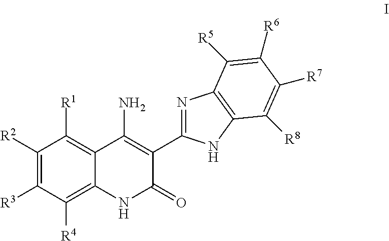

wherein:

R¹, R², R³, and R⁴ may be the same or different and are independently selected from H, Cl, Br, F, I, —OR¹⁰ groups, —NR¹¹R¹² groups, substituted or unsubstituted primary, secondary, or tertiary alkyl groups, substituted or unsubstituted aryl groups, substituted or unsubstituted alkenyl groups, substituted or unsubstituted alkynyl groups, substituted or unsubstituted heterocyclyl groups, or substituted or unsubstituted heterocyclylalkyl groups;

R⁵, R⁶, R⁷, and R⁸ may be the same or different and are independently selected from H, Cl, Br, F, I, —OR¹³ groups, —NR¹⁴R¹⁵ groups, —SR¹¹ groups, substituted or unsubstituted primary, secondary, or tertiary alkyl groups, substituted or unsubstituted aryl groups, substituted or unsubstituted alkenyl groups, substituted or unsubstituted alkynyl groups, substituted or unsubstituted heterocyclyl groups, substituted or unsubstituted heterocyclylalkyl groups, substituted or unsubstituted alkoxyalkyl groups,

substituted or unsubstituted aryloxyalkyl groups, or substituted or unsubstituted heterocyclyloxyalkyl groups;

R¹⁰ and R¹³ may be the same or different and are independently selected from substituted or unsubstituted alkyl groups, substituted or unsubstituted aryl groups, substituted or unsubstituted heterocyclyl groups, substituted or unsubstituted heterocyclylalkyl groups, substituted or unsubstituted alkoxyalkyl groups, substituted or unsubstituted aryloxyalkyl groups, or substituted or unsubstituted heterocyclyloxyalkyl groups;

R¹¹ and R¹⁴ may be the same or different and are independently selected from substituted or unsubstituted alkyl groups, substituted or unsubstituted aryl groups, or substituted or unsubstituted heterocyclyl groups;

R¹² and R¹⁵ may be the same or different and are independently selected from substituted or unsubstituted alkyl groups, substituted or unsubstituted aryl groups, or substituted or unsubstituted heterocyclyl groups; and

R¹⁶ is selected from substituted or unsubstituted alkyl groups, substituted or unsubstituted aryl groups, or substituted or unsubstituted heterocyclyl groups.

The dual KIT inhibitor and FGFR inhibitor compound may also be selected from a compound of Formula II or a tautomer thereof, a pharmaceutically acceptable salt of the compound, a pharmaceutically acceptable salt of the tautomer, or a mixture thereof, wherein the compound of formula II has the following formula:

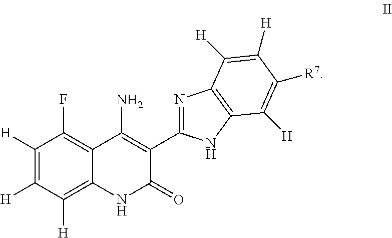

wherein:

R⁷ is a substituted or unsubstituted heterocyclyl group. In some embodiments, R⁷ is a substituted or unsubstituted heterocyclyl group selected from a substituted or unsubstituted piperidinyl group, piperazinyl group, or morpholinyl group. In other embodiments, R⁷ is a substituted or unsubstituted N-alkyl piperazinyl group. In further embodiments, R⁷ is a substituted or unsubstituted N-alkyl piperazinyl group and the alkyl group of the N-alkyl piperazinyl comprises from 1 to 4 carbon atoms.

The dual KIT inhibitor and FGFR inhibitor compound may also be selected from a compound of Formula III or a tautomer thereof, a pharmaceutically acceptable salt of the compound, a pharmaceutically acceptable salt of the tautomer, or a mixture thereof, wherein the compound of formula III has the following formula:

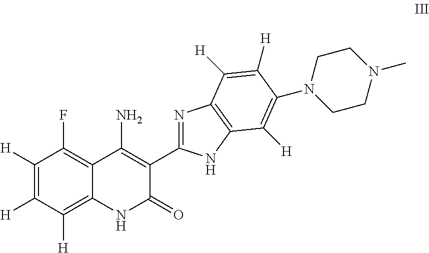 [SMILES: CN1CCN(c2ccc3nc(-c4c(N)c5c(F)cccc5[nH]c4=O)[nH]c3c2)CC1]

Compounds of Formula III include 4-amino-5-fluoro-3-[5-(4-methylpiperazin-1-yl)-1H-benzimidazol-2-yl]quinolin-2(1H)-one (Compound A) and (4-amino-5-fluoro-3-[6-(4-methylpiperazin-1-yl)-1H-benzimidazol-2-yl]quinolin-2(1H)-one) (dovitinib).

In a preferred embodiment, the pharmaceutical combination of the present invention includes at least one compound of Formula I or a tautomer thereof, compound of Formula II or a tautomer thereof, compound of Formula III or a tautomer thereof, a pharmaceutically acceptable salt of the compound, a pharmaceutically acceptable salt of the tautomer, or a mixture thereof that is Compound A.

In another preferred embodiment, the pharmaceutical combination of the present invention includes at least one compound of Formula I or a tautomer thereof, compound of Formula II or a tautomer thereof, compound of Formula III or a tautomer thereof, a pharmaceutically acceptable salt of the compound, a pharmaceutically acceptable salt of the tautomer, or a mixture thereof that is dovitinib.

The dual KIT inhibitor and FGFR inhibitor compounds of Formula I or a tautomer thereof, compounds of Formula II or a tautomer thereof, compounds of Formula III or a tautomer thereof, a pharmaceutically acceptable salt of the compound, a pharmaceutically acceptable salt of the tautomer, or a mixture thereof; formulations of same, and methods for preparing same are described in, for example, WO2002/222598, WO2003/087095, WO2005/046589, WO2006/127926, WO2006/124413, WO2007/064719, WO2009/115562 and WO2012/001074 which are hereby incorporated by reference in entirety.

The compound of the invention may be administered in free form or in pharmaceutically acceptable salt form.

A “pharmaceutically acceptable salt”, as used herein, unless otherwise indicated, includes a salt with an inorganic base, organic base, inorganic acid, organic acid, or basic or acidic amino acid. As salts of inorganic bases, the invention includes, for example, alkali metals such as sodium or potassium; alkaline earth metals such as calcium and magnesium or aluminum; and ammonia. As salts of organic bases, the invention includes, for example, trimethylamine, triethylamine, pyridine, picoline, ethanolamine, diethanolamine, and triethan-olamine. As salts of inorganic acids, the instant invention includes, for example, hydrochloric acid, hydroboric acid, nitric acid, sulfuric acid, and phosphoric acid. As salts of organic acids, the instant invention includes, for example, formic acid, acetic acid, trifluoroacetic acid, fu-maric acid, oxalic acid, tartaric acid, maleic acid, lactic acid, citric acid, succinic acid, malic acid, methanesulfonic acid, benzenesulfonic acid, and p-toluenesulfonic acid. As salts of basic amino acids, the instant invention includes, for example, arginine, lysine and ornithine. Acidic amino acids include, for example, aspartic acid and glutamic acid.

The monolactate salt of the compound of Formula I exists in a variety of polymorphs, including, e.g., the monohydrate form and the anhydrous form. Polymorphs occur where the same composition of matter (including its hydrates and solvates) crystallizes in a different lat-tice arrangement resulting in different thermodynamic and physical properties specific to the particular crystalline form.

Additional pharmaceutically acceptable salts of Compound A and dovitinib suitable for the present invention include the salts, as disclosed in W02005/04658.

In one embodiment, dovitinib is administered daily to a suitable subject in single or divided doses at an effective dosage in the range of about 0.001 to about 100 mg per kg body weight per day, preferably about 1 mg/kg/day to about 35 mg/kg/day, in single or divided doses. For a 70 kg human, this would amount to about 0.07 to 2.45 g/day, preferably about 0.05 to about 1.0 g/day.

The following aspects of the invention are of particular importance:
(1.) A method of treating GIST in a human patient comprising administering to the human patient in need thereof a dose effective against GIST of a combination (a) a c-kit inhibitor and a KIT inhibitor or (b) a dual KIT inhibitor and FGFR inhibitor or FGFR inhibitor, or a pharmaceutically acceptable salt thereof, respectively, especially wherein the c-kit inhibitor is selected from imatinib, nilotinib and masitinib, or, respectively, a pharmaceutically acceptable salt thereof.
(2.) A method of treating GIST in a human patient comprising administering to the human patient in need thereof a dose effective against GIST, wherein the GIST is progressing after imatinib therapy or after imatinib and sunitinib therapy.
(3.) A combination comprising (a) a c-kit inhibitor and (b) a FGFR inhibitor or a pharmaceutically acceptable salt thereof, respectively, for the treatment of GIST.

For the purposes of the present invention, a combination comprising (a) a c-kit inhibitor and (b) a FGFR inhibitor is preferably selected from

(1) imatinib or a pharmaceutically acceptable salt thereof and COMPOUND A or a pharmaceutically acceptable salt thereof,
(2) imatinib or a pharmaceutically acceptable salt thereof and dovitinib or a pharmaceutically acceptable salt thereof.

The structure of the active agents identified by generic or trade names may be taken from the actual edition of the standard compendium “The Merck Index” or from databases, e.g. Patents International (e.g. IMS World Publications). The corresponding content thereof is hereby incorporated by reference.

Unless mentioned otherwise, c-KIT inhibitors, dual KIT inhibitor and FGFR inhibitors and FGFR inhibitors are used in a dosage as either specified in the product information of a product comprising such inhibitor for the treatment of a proliferative disorder, or, especially if such product information is not available, in a dosage which is determined in dose finding studies.

Suitable clinical studies in human patients are, for example, open label non-randomized, studies in patients with GIST progressing after imatinib first line therapy. Such studies prove in particular superiority of the claimed method of treatment compared to treatments with one of the components of the treatment schedule alone. The beneficial effects on GIST can be determined directly through the results of these studies (e.g. RFS or progression free survival—PFS) or by changes in the study design which are known as such to a person skilled in the art.

### EXAMPLES

The following Example illustrates the invention described above, but is not, however, intended to limit the scope of the invention in any way. Other test models known as such to the person skilled in the pertinent art can also determine the beneficial effects of the claimed invention.

### Example 1

### FGF Receptor 1 (FGFR1) and FGF2 Expression in Primary GISTs

### Cell Lines and Culture

GIST882, GIST48 and GIST430 cell lines were obtained from the Brigham and Women's Hospital, Boston, Mass. GIST882 was established from an untreated human GIST with a homozygous missense mutation in KIT exon 13, encoding a K642E mutant KIT protein (Tuveson D A, Willis N A, et al. Oncogene 2001; 20: 5054-5058). GIST48 and GIST430 were established from GISTs that has progressed after initial clinical response to imatinib treatment (Bauer S, Yu L K, Demetri G D, Fletcher J A. Cancer Res 2006; 66: 9153-9161). GIST48 has a primary homozygous exon 11 missense mutation (V560D) and a secondary heterozygous exon 17 missense mutation (D820A). GIST430 has a primary heterozygous exon 11 in-frame deletion and a secondary heterozygous exon 13 missense mutation (V654A). GIST-T1 was obtained from Kochi Medical School, Kochi, Japan. It was established from a metastatic human GIST with a heterozygous deletion of 57 bases in exon 11 of KIT (Taguchi T, Sonobe H, Toyonaga S, et al. Lab Invest 2002; 82: 663-665).

GIST882 cells were cultured in RPMI-1640 (ATCC Catalog #30-2001) supplemented with 15% FBS and 1% L-glutamine, GIST48 cells in F10 (Gibco/Invitrogen Catalog #11550-043) supplemented with 15% FBS, 0.5% Mito+ (BD Bioscience Catalog #355006), 1% BPE (BD Bioscience/Fisher Catalog#354123) and 1% L-glutamine, GIST430 cells in IMEM (Gib-co/Invitrogen Catalog #12440-053) supplemented with 15% FBS and 1% L-glutamine, and GIST-T1 cells in DMEM (Gibco/Invitrogen Catalog #11965) supplemented with 10% FBS.

### Cell Viability Assay

Imatinib and dovitinib were dissolved in DMSO as a 10 mM stock, and subsequently diluted with media to make a series of working solutions at concentrations (μM) of 0, 0.02, 0.05, 0.16, 0.49, 1.48, 4.44, 13.3 and 40. 10,000 cells suspended in 80 μl media were seeded into each well of a 96-well cell-culture plate and grown for 24 hours prior to treatment. 10 μl of 60 μg/mL heparin (Sigma Catalog # H3149) was added to each well, and then 10 μl of 50 μg/mL FGF2 (R&D Catalog #233-FB/CF) or media was added to each well of the plates. 10 μl of each of the compound dilutions described above and 10 μl of media were added to wells to a final volume 120 μl such that all pair-wise combinations as well as the single agents were represented. Cells were incubated for 72 hrs at 37° C. in a 5% CO2 incubator following compound addition. Cell proliferation was measured using the CellTiter-Glo luminescent cell viability assay (Promega catalog # G755B) and Victor4 μlate reader (Perkin Elmer). Synergy scores and CI₇₀ calculations were determined as described elsewhere (Lehar J, Krueger A S, et al. Nat Biotechnol 2009; 27: 659-666).

### Western Blotting

Protein lysates were prepared from cell monalayers using RIPA buffer (Cell Signaling Technology Catalog #9806) according to the procedure described by the manufacturer. Antibod-ies to detect phospho-KIT (Catalog #3073S), total KIT (Catalog #3308), phospho-AKT S473 (Catalog #4058), total AKT (Catalog #9272), phospho-ERK (Catalog #9101), total ERK (Catalog #9107) and phospho-FRS2 (Catalog #3864) were purchased from Cell Signaling Technology. Antibody to GAPDH (Catalog # MAB374) was purchased from Millipore and anti-FRS2(H-91) (Catalog #sc-8318) from Santa Cruz. Bound antibody was detected using the LI-COR Odyssey Infrared Imaging System.

### Results

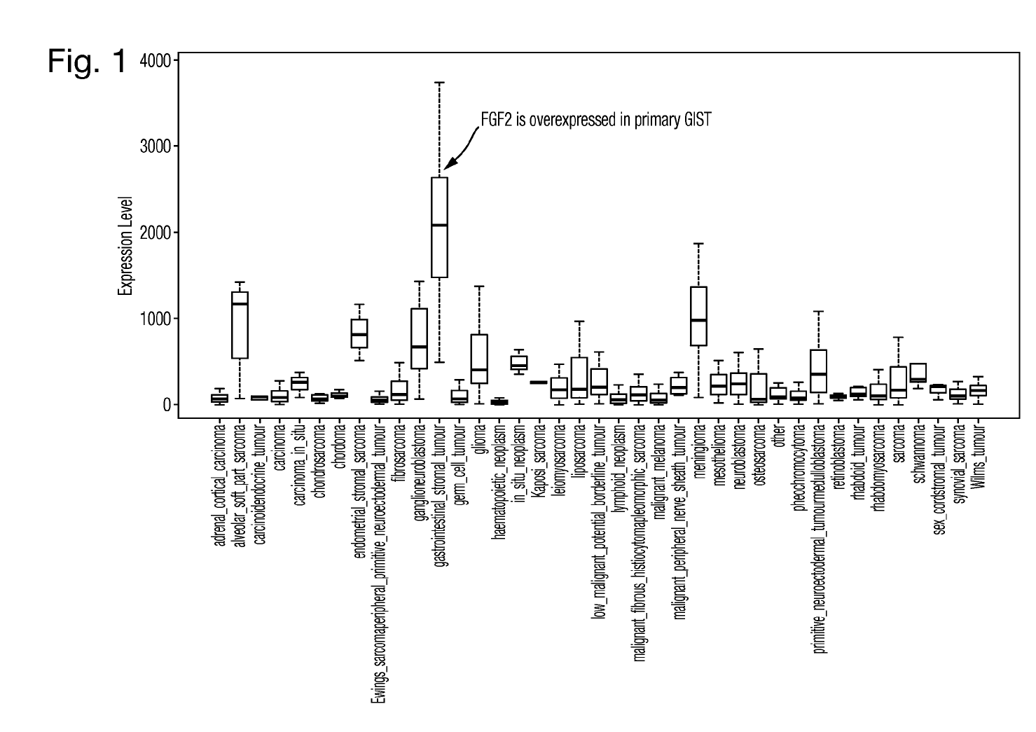

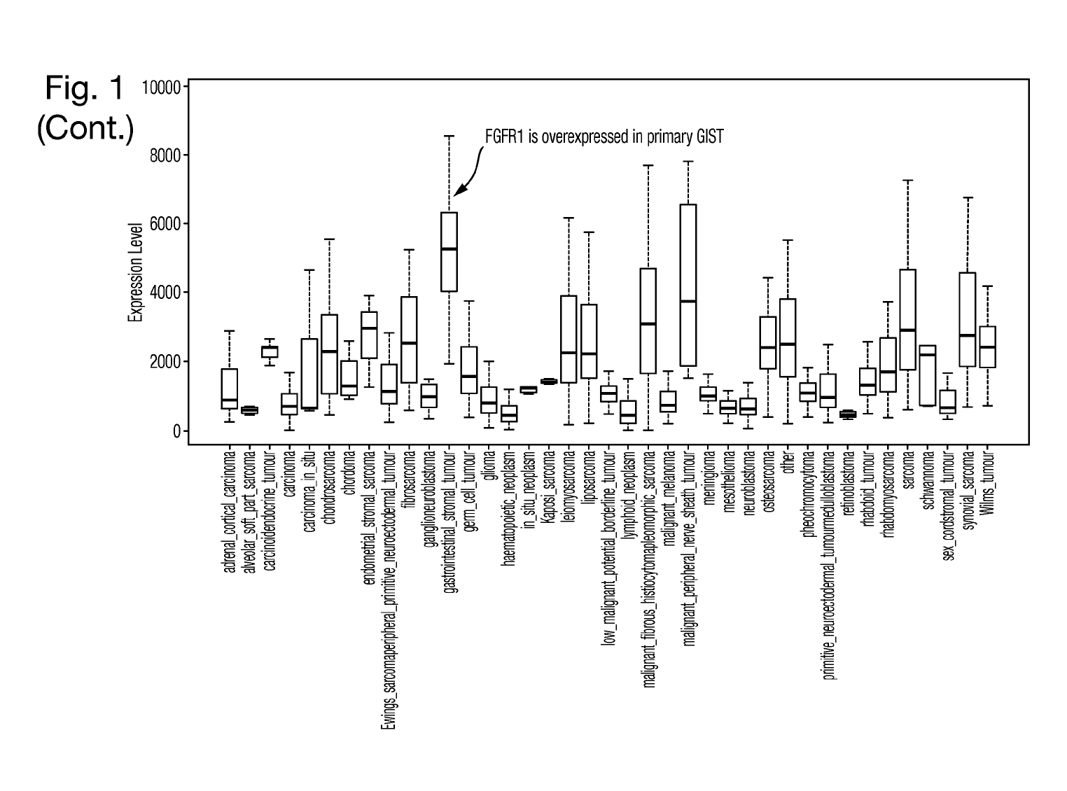

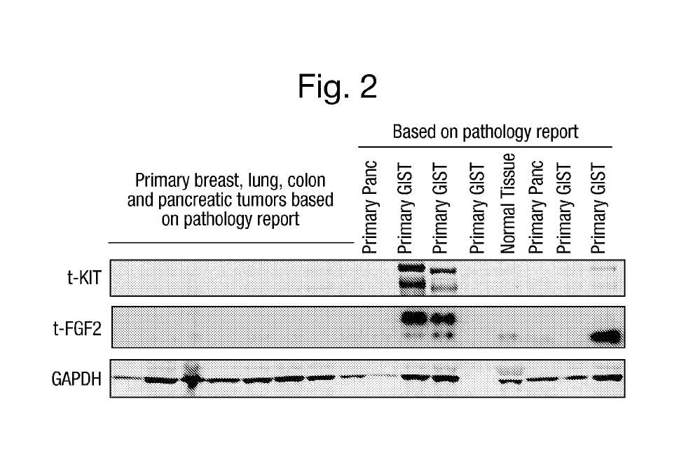

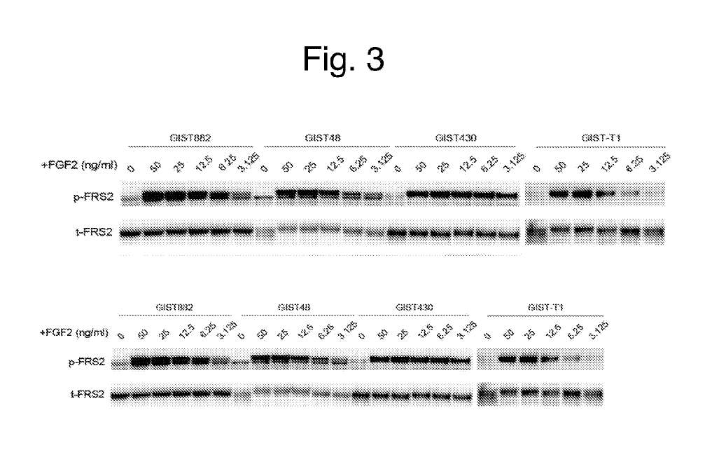

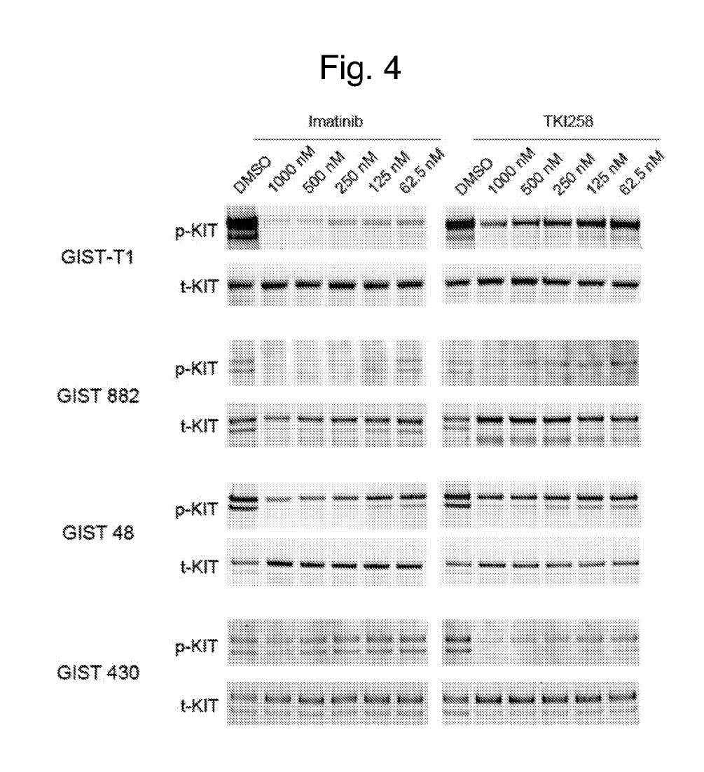

Novartis OncExpress database contains both internally and publically deposited expression data for 30,094 primary tumors, including 110 GIST samples, profiled by Affymetrix Human Genome U133A or U133 Plus 2.0 arrays. In addition to the known GIST-specific genes, such as KIT, ETV1 and PRKCQ, FGF2 and its receptor FGFR1 showed the highest average expression levels in GIST among 41 tumor types included in this dataset (FIG. 1), suggesting that FGFR pathway could be a survival pathway in GIST. FGF2 was also found to be over-expressed at the protein level in primary GISTs (FIG. 2). FGFR1, but not FGF2, is over-expressed in GIST cell lines. However, the FGFR signaling pathway was activated when various concentrations of exogenous FGF2 was added (FIG. 3). KIT inhibition of Imatinib and dovitinib was also measured by Western blot in the GIST cell lines (FIG. 4).

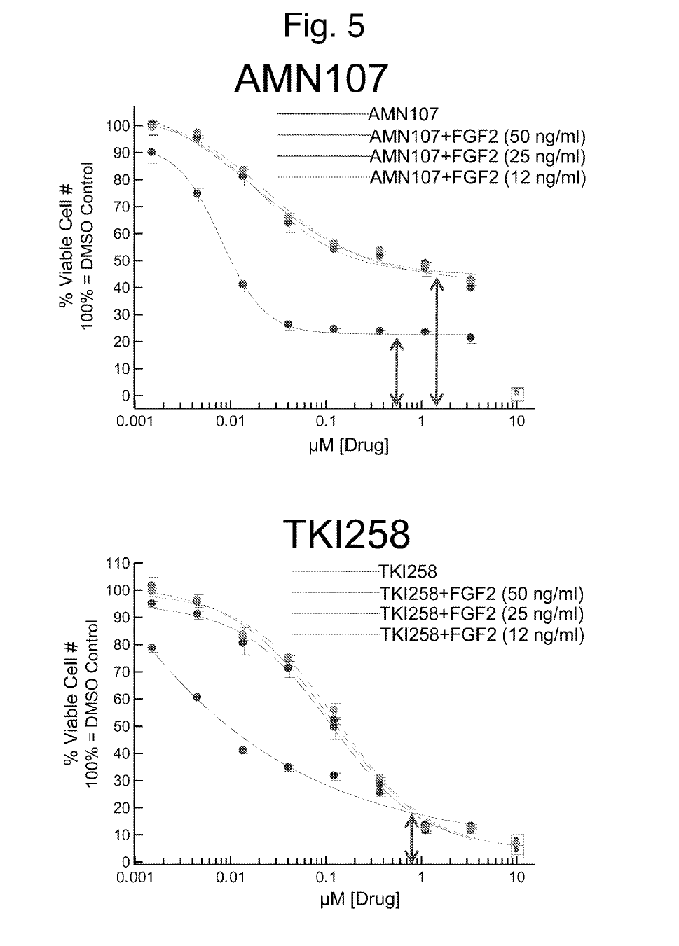

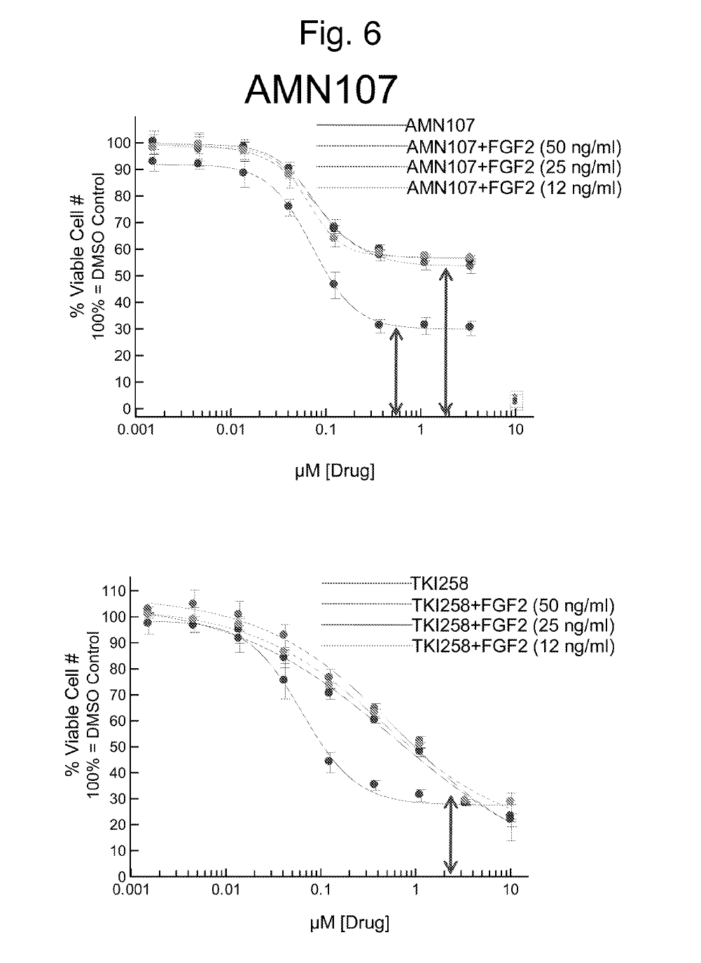

GIST-T1 and GIST882 are sensitive to KIT inhibition achieved by nilotinib treatment (FIGS. 5 and 6, upper panels). However, these two cell lines were shown to be less sensitive to KIT inhibition in the presence of added FGF2 with the GI₅₀ values shifted greater than 10 fold (FIGS. 5 and 6, upper panels), suggesting that FGFR signaling can function as a survival pathway once activated. Therefore, combining a KIT inhibitor and a potent FGFR inhibitor should enhance the growth inhibition in the GIST cell lines.

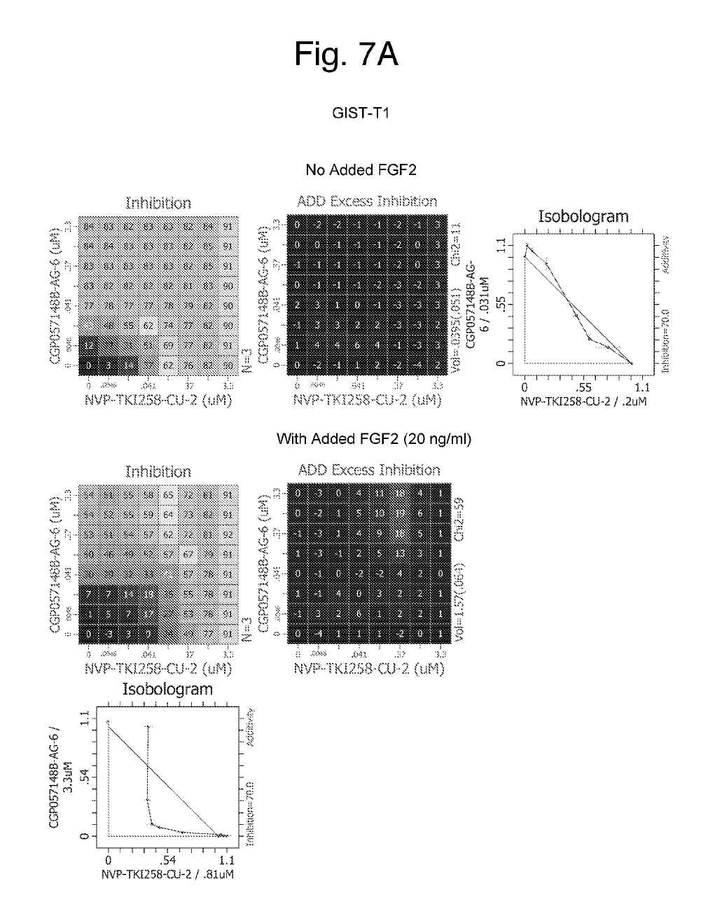

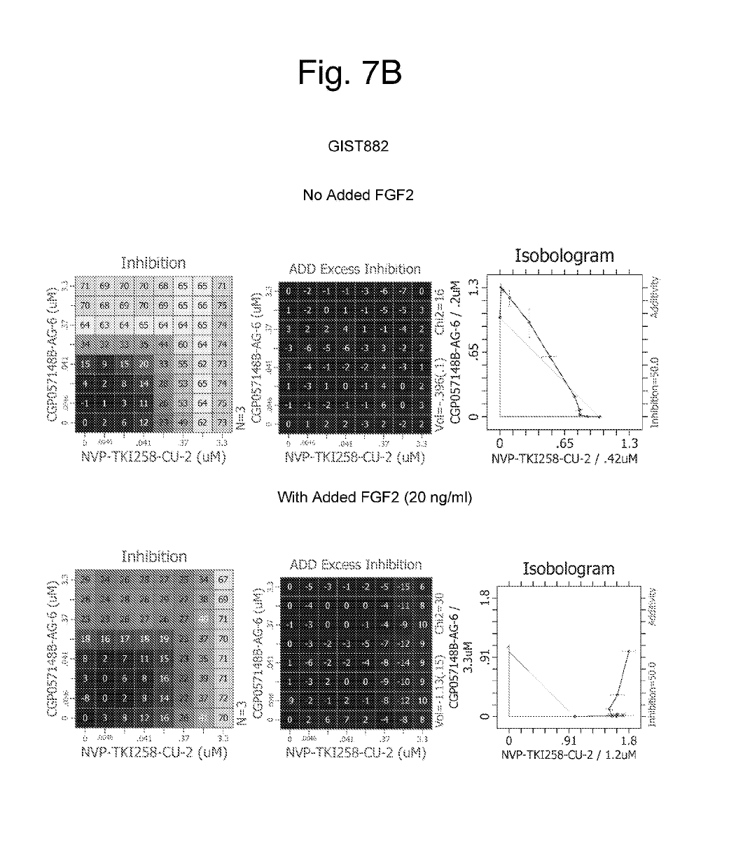

Dovitinib is an orally active, potent and selective inhibitor of FGFRs in addition to being a dual KIT inhibitor and FGFR inhibitor. When GIST-T1 and GIST882 were treated with dovitinib in the presence of added FGF2, the maximum inhibition was restored to levels comparable to the absence of FGF2, suggesting that dovitinib, as a dual KIT and FGFR inhibitor, inhibited both KIT and FGFR pathway (FIGS. 5 and 6, lower panels). To determine the single agent and combination effects of combining the FGFR inhibitor dovitinib and the KIT inhibitor imatinib (CGP057148B) on the growth inhibition of GIST cells, the proliferation responses for cells treated with dose ranges of each compound alone and pair-wise combinations for 3 days were compared. As a single agent, imatinib was efficacious in inhibiting GIST-T1 and GIST882 growth in the absence of FGF2 (FIG. 7). In the presence of added FGF2, these two cell lines were less sensitive to imatinib treatment (FIG. 7), similar to the results shown in FIGS. 5 and 6. Dovitinib was effective in inhibiting GIST-T1 and GIST882 regardless of the presence or absence of added FGF2, consistent with the findings shown in FIGS. 5 and 6 (FIG. 7). A dovitinib combination with a KIT inhibitor (imatinib) resulted in weak combination effects in the presence of FGF2 in GIST cells due to the fact that dovitinib was able to inhibit both KIT and FGFR pathway. However, imatinib is more potent KIT inhibitor than dovitinib in GIST-T1, GIST882 and GIST48 (FIG. 4), suggesting that combining imatinib with dovitinib may still have clinical benefits. Combination effects were shown in FIG. 7 and determined by combination indices at a 70% inhibitory effect (CI₇₀) that measure dose shifting yielding 70% growth inhibition and by synergy scores that measure overall synergy observed across the entire dose matrixes (Lehar J, Krueger A S, al. Nat Biotechnol 2009; 27: 659-666).

Also the combination of imatinib and dovitinib shows synergy in GIST cell lines even in the presence of FGF2 (FIG. 7). The effects of dovitinib and of imatinib have been evaluated both as single agents and in combination in patient-derived GIST882 (expressing K642E mutant KIT), GIST430 (expressing ex11del/V654A KIT) and GIST-T1 (expressing ex11del KIT) cell lines. When the antiproliferative effects of imatinib and dovitinib were evaluated in combination, growth suppression was observed in excess of the percent inhibition achieved by imatinib or dovitinib single agent treatment in GIST882 and GIST430 cell-lines.

TABLE 1

| Imatinib | | | |
|---|---|---|---|
| combined | | | |
| with | GIST882 | GIST-T1 | GIST430 |
| | Synergy Score for inhibition of GIST cell proliferation | | |
| Dovitinib | 0.321 | 0.422 | 0.635 |
| | Combination Index for 70% | | |
| | inhibition of GIST cell proliferation | | |
| Dovitinib | 0.965 | 0.835 | 0.624 |

Synergy is quantified either as the ‘weighted’ Synergy Score, S (where S≦1 indicates either some additivity or no cooperativity or, S>1 suggests of some synergy and S>2 indicates significant synergy) or as Combination Indices, CI (where CI=1 indicates dose additivity, CI<0.5 indicates “real” synergy (2× dose shift), CI<0.3 indicates “useful” synergy (3× shift) and CI<0.1 indicates “strong” synergy (10× shift). Significant assessments of synergy are indicated in bold-type.

### Example 2

### Single-Arm Dose-Finding Phase Ib Study of Imatinib in Combination with the Dual KIT Inhibitor and FGFR Inhibitor Dovitinib in Patients with Gastrointestinal Stromal Tumor (GIST) Who Failed Prior Therapy with Imatinib and Sunitinib

Inclusion criteria:
1. Male or female patients 18 years of age
2. WHO performance status (PS) of 0-2
3. Histologically confirmed diagnosis of GIST that is unresectable or metastatic
4. Available tissue specimen:
   Dose-escalation cohorts: patients must have available archival tumor tissue which can be shipped during the course of the study
   Dose-expansion cohort: patients must have available archival tumor tissue which can be shipped during the course of the study and must agree to a fresh pre-treatment biopsy.
5. Failed prior therapy with imatinib followed by sunitinib for the treatment of unresectable or metastatic GIST. Note the following specific criteria for the two phases of the trial:
   Dose-escalation cohorts: patients who failed prior therapy with imatinib and then have failed therapy with sunitinib. Treatment failure may be due to either disease progression on therapy (both imatinib and sunitinib) or intolerance to therapy (sunitinib).
   Dose-expansion cohort: patients must have documented disease progression on both imatinib and sunitinib. In addition, patients may have had no more than two lines of prior therapy (i.e. treatment with imatinib followed by treatment with sunitinib).

1. A pharmaceutical combination comprising (a) a c-kit inhibitor and (b) a dual KIT inhibitor and FGFR inhibitor or FGFR inhibitor, or a pharmaceutically acceptable salt thereof, respectively, for the treatment of GIST.

2. The pharmaceutical combination according to claim 1, wherein the c-kit inhibitor is selected from imatinib, nilotinib and masitinib, or, respectively, a pharmaceutically acceptable salt thereof.

3. The pharmaceutical combination according to claim 2, wherein the dual KIT inhibitor and FGFR inhibitor is 4-amino-5-fluoro-3-[5-(4-methylpiperazin-1-yl)-1H-benzimidazol-2-yl]quinolin-2(1H)-one or a pharmaceutically acceptable salt or tautomer thereof.

4. Method of treating GIST in a human patient comprising administering to the human patient in need thereof a dose effective against GIST of a combination (a) a c-kit inhibitor and (b) a dual KIT inhibitor and FGFR inhibitor or FGFR inhibitor, or a pharmaceutically acceptable salt thereof, respectively.

5. The method according to claim 4, wherein the c-kit inhibitor is selected from imatinib, nilotinib and masitinib, or, respectively, a pharmaceutically acceptable salt thereof.

6. The method according to claim 4, wherein the dual KIT inhibitor and FGFR inhibitor is 4-amino-5-fluoro-3-[5-(4-methylpiperazin-1-yl)-1H-benzimidazol-2-yl]quinolin-2(1H)-one or a pharmaceutically acceptable salt or tautomer thereof.

7. The method according to claim 4, wherein the GIST is progressing after imatinib therapy.

8. The method according to claim 4, wherein the GIST is progressing after imatinib and sunitinib therapy.

9. The method according to claim 5, wherein imatinib is applied in a daily dose between 300 and 600 mg.
